(19) United States
(12) Patent Application Publication
(10) Pub. No.: US 2008/0038204 A1
(43) Pub. Date: Feb. 14, 2008

(54) Whole Body Imaging Using Portable Observation Systems

(76) Inventors: Meng Yang, San Diego, CA (US); Ping Jiang, San Diego, CA (US)

Correspondence Address: MORRISON & FOERSTER LLP, 12531 HIGH BLUFF DRIVE, SUITE 100, SAN DIEGO, CA, 92130-2040, US

(21) Appl. No.: 10/587,528

(22) PCT Filed: Jan. 26, 2005
(86) PCT No.: PCT/US05/03001

§ 371 Date: Jul. 5, 2007

Related U.S. Application Data
(60) Provisional application No. 60/539,464, filed on Jan. 26, 2004.
(60) Provisional application No. 60/540,599, filed on Jan. 29, 2004.

(51) Int. Cl. A61B 5/00

(52) U.S. Cl. 424/9.6

(57) ABSTRACT

Methods to image a fluorescent protein in intact subjects using simple portable instruments are described.

### CROSS-REFERENCE TO RELATED APPLICATIONS

This application claims benefit under 35 U.S.C. § 119(e) to U.S. application Ser. No. 60/539,464 filed 26 Jan. 2004 and U.S. Ser. No. 60/540,599 filed 29 Jan. 2004. The contents of these documents are incorporated herein by reference.

### TECHNICAL FIELD

The invention relates to methods of imaging based on fluorescent protein emission in vivo using portable instruments. Cells containing fluorescent protein are observed within a subject using simple external imaging techniques. The portable observation equipment may be configured to screen large numbers of subjects containing the fluorescent protein.

### BACKGROUND ART

Whole-body imaging technology has been used to monitor “tracer molecules” in the intact body. For example, Brenner, et al., studied the diagnostic value of iodine-123-2-hydroxy-3-iodo-6-methoxy-N-[(1-ethyl-2-pyrrolidinyl)methyl]benzamide (IBZM) whole-body imaging in comparison to thallium-201 scintigraphy in patients with metastatic malignant melanoma (Brenner, et al., Eur. J. Nucl. Med., (1999)26:1567-1571). Benard, et al., conducted clinical evaluation of processing techniques for attenuation correction with ¹³⁷Cs in whole-body PET imaging (Benard, et al., J. Nucl. Med., (1999) 40:1257-1263). Jerusalem, et al., showed that whole-body positron emission tomography using ¹⁸F-fluorodeoxyglucose for posttreatment evaluation in Hodgkin's disease and non-Hodgkin's lymphoma has higher diagnostic and prognostic value than classical computed tomography scan imaging (Jerusalem, et al., Blood, (1999) 94:429-433). Eustace, et al., discussed practical issues, clinical applications, and future directions of whole-body MR imaging (Eustace, et al., Magn. Reson. Imaging Clin. (N. Am.) (1999) 7:209-236). Engelson, et al., studied fat distribution in HIV-infected patients reporting truncal enlargement quantified by whole-body magnetic resonance imaging (Engelson, et al., Am. J. Clin. Nutr. (1999) 69:1162-1169). Valk, et al., used whole-body positron emission tomography (PET) imaging with [¹⁸F]fluorodeoxyglucose in management of recurrent colorectal cancer (Valk, et al., Arch. Surg. (1999) 134:503-511). Saunders, et al., evaluated fluorine-18-fluorodeoxyglucose whole body positron emission tomography imaging in the staging of lung cancer (Saunders, et al., Ann. Thorac. Surg. (1999) 67:790-797).

The ability to visualize gene expression, infection, and tumor metastasis in vivo is clearly a valuable technology useful in understanding both the progression of inherent sequelae and in devising mechanisms for altering this progression. Early forms of this technology that employ detection of emitted light, as opposed to more complex systems such as those described above, were invasive and required either excision of tissue or providing, for example, subcutaneous windows with semitransparent materials.

Relatively recently, techniques have been devised to use light emission to observe the internal behavior of tumor cells or other cells of interest in an intact animal. For example, PCT publication WO 97/18841 describes the use of the lux operon to modify Salmonella so that these infectious agents become bioluminescent. The course of infection could be followed in the intact animal without the need for external excitation of fluorescence, but the observations require immobilization of the subject in order to provide sufficient image intensity. Thus, the animals must be immobilized in a light-tight box and images created with a charge-coupled-device camera with a two-stage microchannel intensifier head attached to an Argus 50 image processor. The method requires maintaining the subject in an immobilized condition, measuring photon emission from the light-generating moiety, localized in the subject, with a photodetector device until an image of photon emission can be constructed; and detecting the image through an opaque tissue of said mammal. Complex technology is required to obtain meaningful images.

PCT publication WO 98/49336 describes metastasis models which use green fluorescent protein (GFP) as a marker. As described in this publication, GFP is a commonly used designation for fluorescent proteins which emit a variety of colors, not just green. The metastases from implanted tumors could be observed in real time without the necessity for immobilizing the subjects due to the high intensity fluorescence available from this protein. Observations in the intact animal were observed, in this instance, through fluorescent microscopy, again requiring fairly complex technology for observation. Similar techniques were described in the subsequent PCT publication WO 00/40274. These techniques were extended to the observation of gene expression using green fluorescent protein in WO 01/71009 and to the progression of infection in WO 2004/016766.

This approach is further described by Yang, et al., Proc. Natl. Acad. Sci. (USA) (2000) 97:1206-1211), which discloses, in real time, images of fluorescent tumors growing and metastasizing in live mice. The whole-body optical imaging system is external and noninvasive. It affords unprecedented continuous visual monitoring of malignant growth and spread within intact animals. Yang, et al., have established new human and rodent tumors that stably express very high levels of the Aequorea victoria green fluorescent protein (GFP) and transplanted these to appropriate animals. Whole-body optical images showed, in real time, growth of the primary colon tumor and its metastatic lesions in the liver and skeleton. Imaging was with either a trans-illuminated epifluorescence microscope or a fluorescence light box and thermoelectrically cooled color charge-coupled device camera. The depth to which metastasis and micrometastasis could be imaged depended on their size. The apparatus used for observation was, however, complex.

For visualization at high magnification, Leica fluorescence stereo microscope, model LZ12, equipped with a 50-W mercury lamp, was used. Selective excitation of GFP was produced through a D425/60 band-pass filter and 470 DCXR dichroic mirror. Emitted fluorescence was collected through a long-pass filter GG475 (Chroma Technology, Brattleboro, Vt.) on a Hamamatsu C5810-3-chip cooled color charge-coupled device camera (Hamamatsu Photonics Systems, Bridgewater, N.J.). Images were processed for contrast and brightness and analyzed with the use of IMAGE PRO PLUS 3.1 software (Media Cybernetics, Silver Springs, Md.). Images of 1,024×724 pixels were captured directly on an IBM PC or continuously through video output on a high-resolution Sony VCR model SLV-R-1000 (Sony, Tokyo).

Imaging at lower magnification that visualized the entire animal was carried out in a light box illuminated by blue light fiber optics (Lightools Research, Encinitas, Calif.) and imaged by using the thermoelectrically cooled color charge-coupled device camera, as described above.

Further, the intensity of GFP fluorescence is measured to account for variations in the exciting illumination with time and across the imaging area. These factors are corrected for by using the intrinsic red fluorescence of mouse skin as a base line to correct the increase over intrinsic green fluorescence caused by GFP. This can be done because there is relatively little red luminence in the GFP radiance. Consequently, the green fluorescence was calculated relative to red based on red and green channel composition in the skin image. A ratio (γ) of green to red channels was determined for each pixel in the image of skin without and with GFP. Values of γ for mouse skin throughout the image in the absence of GFP were fairly constant, varying between 0.7 and 1.0. The contribution of GFP fluorescence from within the animal increased the green component relative to red, which was reflected in higher γ values. The total amount of GFP fluorescence was approximated by multiplying the number of pixels in which value γ was higher than 1 times the γ value of each pixel. Such a product roughly corresponds to the integral GFP fluorescence [I′_{GFP}] above the maximum value of γ for skin without GFP. The number of pixels in mouse skin images with γ value>1.0 without GFP was less than 0.02% and increased with GFP expression. The value of [I′_{GFP}] is shown as a function of time after virus injection in FIGS. 1A and 1B for brain and liver respectively.

Images of the various organs were compared when taken at high magnification on live intact animals or similar organs viewed directly after death and dissection. The images show the distribution of gene expression in the various organs. In all cases, the images made externally are similar to those of the exposed organs.

When the live animal was viewed in a light box, it was also possible to monitor the expression of the gene, thus permitting a real time observation of the living animal and expression as it occurs in this animal. For example, a light box determination of expression of the GFP in nude mouse liver taken at 72 hours clearly shows this result. Similar results are observed in the nude mouse brain 24 hours after gene delivery.

Weissleder and colleagues infused tumor-bearing animals with probes that fluoresce at an infrared frequency when activated by protease activity (Nat. Biotechnol. (1999) 17:375-378). Tumors with appropriate proteases activated the probes and could be seen externally. This system is limited by very high liver-to-tumor background fluorescence, which means that metastasis to the liver—among the most important metastatic sites—cannot be studied. Furthermore, the time limit for studies was 96 hours, so growth and efficacy studies were not possible. Tumors must have appropriate protease activity to be detectable, and the probes must be delivered selectively to the tumor.

It is apparent that the techniques of the prior art, while useful, require complex equipment and do not lend themselves to rapid evaluation of a multiplicity of subjects in short periods of time. The present invention solves these problems by utilizing portable equipment that can readily be adapted to multiple observations.

### DISCLOSURE OF THE INVENTION

The invention relates to methods of imaging fluorescent protein using portable instruments. The fluorescent protein(s) are observed within a subject using external imaging techniques. By using a portable excitation light source (such as a flashlight) with an attached first filter for the excitation light source and a second filter calibrated to receive the emitted light, real time observations can be made on one or a multiplicity of subjects.

This observation technique can be applied in a multiplicity of contexts—for following the growth and metastasis of tumors, for following the progress of infection, for following gene expression and to evaluate factors that influence each of these processes. Thus, the observation may be made on experimental animals which serve as tumor models or models of infection and these are used as systems for evaluating treatment protocols as well as observing the effect of various stimuli on metabolic function.

The appropriate matching of excitation filters and observation filters on the portable equipment permits informative imaging regardless of the context in which the fluorescent signal is observed.

Thus, in one aspect, the invention is directed to a method to visualize a fluorescent protein through the skin of an intact subject which method comprises applying excitation light to said subject using a portable light source with an attached first filter and observing emission from said protein through a second filter. In particular aspects, this visualization is employed to monitor tumor progression and metastasis, observe the effect of various protocols on said progression and metastasis, to monitor gene expression and observe the effect of various stimuli on such expression, and to monitor infection and observe the effects of various treatments and protocols on the progress of said infection.

### MODES OF CARRYING OUT THE INVENTION

The invention relates to methods of in vivo or whole-body imaging of a subject using fluorescent protein as a tracer and using a portable light source for excitation and a portable filter for detection.

A number of suitable fluorescent proteins are available and well known in the art. For example, the Green Fluorescent Protein (GFP) gene, cloned from the bioluminescent jellyfish Aequorea Victoria (Anticancer Res. (1994) 14:85-92), was chosen to satisfy these conditions because it has great utility as a cellular marker (Science (1994) 263:802-805; Nat. Biotechnol. (1996) 14:606-609). GFP cDNA encodes a 283 amino acid monomeric polypeptide with a molecular weight of 27 kDa (Gene (1992) 111:229-233; Nat. Biotechnol. (1996) 14:1252-1256) that requires no other Aequorea proteins, substrates, or cofactors to fluoresce (Biochemistry (1993) 32:1212-1218). Recently, gain-of-function bright mutants expressing the GFP gene have been generated by various techniques (Nature (1995) 373:663-664; Biotechnology (1995) 13:151-154; Gene (1996) 173:33-38; Nat Biotechnol. (1996) 14:315-319; Nat Biotechnol. (1996) 14:315-319) and have been humanized for high expression and signal (J. Virol. (1996) 70:4646-4654). Red fluorescence proteins (RFP) from the Discosoma coral have also been described and should prove useful for in vivo imaging studies (PNAS USA (2000) 97:11990-11995; FEBS Lett. (2000) 479:127-130; Nat Biotechnol. (1999) 17:969-973.

Several groups have selected tumor cell lines to stably express GFP at high levels both in vitro and in vivo. These cells have been transplanted into animals and visualized in situ in fresh tissues (PNAS USA (1997) 94:11573-11576). Furthermore, tumor cells expressing GFP have been visualized with or without subsequent colonization in all the major organs including liver, lung, brain, spinal cord, axial skeleton, and lymph nodes. GFP models of metastatic disease have been developed for lung cancer (Clin Exp Metastasis. (1997) 15:547-552), prostate cancer (Cancer Res. (1999) 59:781-786), melanoma (Clin Cancer Res. (1999) 5:3549-3559), colon cancer (PNAS USA (2000) 97:1206-1211), pancreatic cancer (Clin Exp Metastasis (2000) 18:213-218), breast cancer (Clin Exp Metastasis (1999) 17:537-544), ovarian cancer (Clin Exp Metastasis (1999) 17:417-422), and brain cancer (Neurosurgery (1998) 43:1437-1442). This review shows that tumor cells transfected with the GFP gene are a powerful tool for in vivo visualization of tumor growth, angiogenesis, dormancy, dissemination, invasion, and metastasis.

Table I below lists a number of known fluorescent proteins and relevant excitation and emissions characteristics for the proteins.

TABLE I
Fluorescent Proteins

| | Excitation | Emission |
|---|---|---|
| | maximum | maximum |
| Fluorescent Protein | wavelength | wavelength |
| Cyan fluorescent protein (ECFP)* | 434 | 477 |
| AmCyan | 466 | 488 |
| Green fluorescent protein (EGFP)* | 489 | 508 |
| emeraldGFP | 485-488 | 510 |
| mGFP5er | 405 and 477 | 510 |
| ZsGreen | 496 | 506 |
| Yellow fluorescent protein (EYFP)* | 514 | 527 |
| ZsYellow | 531 | 540 |
| Red fluorescent protein (DsRed)* | 558 | 583 |
| AsRed | 573 | 595 |

*Available from BD Biosciences, San Jose, CA.

The location of these proteins in intact subjects is followed by using a simple optionally handheld excitation light source with an appropriate filter, and observed directly, optionally just by eye, using a filter tuned to transmit the emitted light. For example, Biological Laboratory Equipment, Ltd, Budapest, Hungary (website:bls-ltd.com) makes a number of light weight portable devices suitable for use with the methods of the claimed invention.

Generally, the devices comprise an excitation light source, one or more excitation filters, and one or more barrier filters. The excitation light source and the detection components of the contemplated devices may be part of the same structure or they may be separated into different components.

The excitation light source typically comprises ultra bright blue light emitting diodes (LED's). The excitation frequency will generally range from 400 to 600 nm, achieved using excitation filters with a particular cut off frequency. The barrier filters will typically have a cut off of below 500 nm.

One device for use with the disclosed methods is a goggle assembly that resembles a miner's lamp and provides the user with the ability to move freely about while conducting examinations. The device comprises a light source, typically bright blue LED's, and a barrier filter over the eye pieces. The wide path barrier filters of such a device are suitable for fluorescent protein emission observation. Different filters can be used to observe emissions from different fluorescent proteins.

Another preferred device contemplated for use with the claimed invention is a stationary device under which a plurality of samples may be passed. In a preferred embodiment, this device produces light from a plurality of individual portable and interchangeable light sources. These light sources can be separately aimed, if desired.

The devices useful in the methods of the invention can be designed to permit observations made by the naked eye. Alternatively, the barrier filter can be linked to a camera to enable images to be displayed on a monitor and digitally stored. Images can be processed with standard software and the imaging procedures can be repeated as often as necessary without harming the animal.

In general, the excitation light emitting device and the barrier filter that permits observation are characterized by being “portable”—i.e. sufficiently simple and small that they can be held in the hand and carried around. The excitation light emitting device is similar in size and overall shape to an ordinary flashlight but is provided with a suitable filter to result in the appropriate excitation wavelength(s) reaching the subject. The breadth of the beam is determined by the size of the area of the subject for which observation is desired. The second filter, used for observation, should also be sufficiently small to be hand held, and may be configured to aid convenience. For example, it could be fitted into a goggle, or placed in a frame (analogous to a magnifying glass) or mounted in a support. The last-mentioned option is particularly favored if an image is to be recorded.

While the portable excitation and observation tools are sufficiently simple and small that they can be hand-held, in use they may be mounted on one or more supports and used in a stationary mode. This may be particularly desirable when multiple observations are to be made or multiple subjects used.

One preferred application is monitoring progression and metastasis of tumors. Fluorescent protein-expressing tumors of the colon, prostate, breast, brain, liver, lymph nodes, lung, pancreas, bone, and other organs can be visualized externally by use of a quantitative transcutaneous whole-body fluorescence imaging device that is portable. This technology coupled with in vivo tumor cell transduction can also been used for real-time imaging and targeting of tumor cells to screen compounds for effectiveness against tumor cells. Detailed descriptions of tumor/metastasis models using fluorescent proteins as markers may be found in WO 00/40274 referenced above and incorporated herein by reference. In particular, in one embodiment, viruses with tropisms for tumor cells are employed to deliver one or more exogenous nucleic acid sequences comprising an expression system for a fluorescent protein to a target tumor cell. Retrovirus vectors are a preferred example, such as that described in U.S. Pat. No. 5,998,192, to Russell, et al., hereby incorporated by reference. This patent discusses the use of a recombinant C-type murine leukemia virus (MLV). Adenovirus vectors may also be used such as described in U.S. Pat. No. 6,676,935, to Henderson, et al., incorporated by reference. Any virus that either has a natural or engineered tropism for one or more kinds of cancer cells can be used to delivery exogenous nucleic acid to a target tumor cell. “Tropism” describes viruses that preferentially infect tumor cells over normal cells as well as viruses that infect both normal and tumor cells, but are transcriptionally active only in tumor cells.

Thus, tumor specific viruses can deliver a fluorescent protein to a tumor cell. After the virus genome is introduced into the target tumor cell, the gene or genes encoding the fluorescent protein(s) are transcribed by the cellular machinery and fluorescent protein is produced. Because of the specificity of the virus, tumor cells are preferably labeled.

Other delivery systems such as liposomes or direct application of nucleic acid to tumor cells are also contemplated for use with the present invention. Tumors can also be surgically implanted including orthotopically into subjects for study using the methods described herein.

Other applications are to monitor the progression and nature of the effects of infection by using labeled infectious agents as described in WO 2004/016766, referenced above and incorporated herein by reference. Also useful for application of the method of the invention is monitoring gene expression, especially with respect to response to various external stimuli as described in WO 01/71009, referenced above and incorporated herein by reference.

In all of the foregoing cases, the effects of various protocols, treatments, or stimuli can be monitored by comparing the progress of tumor growth and metastasis, or gene expression, or infection in the presence and absence of the candidate protocols or stimuli. Because the observation methods of the invention are simple and direct, a large number of protocols and stimuli may be evaluated quickly for efficacy and safety.

The nature of the subjects useful in the invention depends on the application. For example, for monitoring tumor progression, immunocompromised rodents or rodents syngeneic with the tumor to be visualized are most convenient. Models of infection include a wider range of laboratory animals, including birds. For evaluation of gene expression, virtually any animal subject, including rodents, rabbits, large domestic animals such as cows and pigs, chickens, and even animals that are typically feral may be used. The limitations on the subject are related to the nature of the metabolic or cellular activity observed, not particularly to the observation system described herein.

The following examples are offered to illustrate but not to limit the invention.

### EXAMPLE 1

### In Vivo Fluorescent Protein Labeling of Tumor Cells

GFP retroviral supernatants are prepared according to Hasegawa, et al. (“In vivo tumor delivery of the green fluorescent protein gene to report future occurrence of metastasis,” Cancer Gene Therapy (2000) 7:1336-1340). Nude mice are prepared with human stomach tumors growing intraperitoneally, also as discussed by Hasegawa, et al. The retroviral supernatants are injected intraperitoneally at days 4 to 10 following implantation of the cancer cells into the mice.

The mice are imaged externally using a GFsP-5 imaging device, which resembles a miner's lamp. Examinations occur every other week so that tumor growth and metastasis formation can be visualized by GFP expression. No normal tissues are transduced by the GFP retrovirus. Two weeks after retroviral GFP delivery, GFP-expressing tumor cells are observed in the gonadal fat, greater omentum, and intestine, indicating that the tumors are efficiently transduced by the GFP gene and can be detected by its expression. Laparotomies are performed to confirm the observations made externally.

### EXAMPLE 2

### High Throughput External Screening of Subjects with Ovarian Tumor Fragments

Thirty nude mice are implanted with GFP-expressing Chinese hamster ovary tumor fragments (CHO-K1-GFP) of about 1 mm³ to screen potential antitumor compounds for efficacy against the tumor tissue. The nude mice are implanted with tumor fragments into the ovarian serosa of nude mice by surgical orthotopic implantation (SOI) and ovarian tumors develop (See, Chishima, et al., Cancer Res. (1997) 57:2042-2047).

The animals, housed in individual cages, are placed on a rotating table and are passed in front of a GFP-Vid-187 (Biological Laboratory Equipment, Ltd, Budapest, Hungary), which comprises a light source fitted upon a standard digital video camera. The tumors, which are strongly fluorescent, are observable on the video gathered by the camera. Images gathered by the camera are analyzed visually and fed into a computer for further analysis.

Experimental animals receive various candidate compounds while the control animals receive saline. During the study, animals receiving a candidate compound that is efficacious against the tumor cells display less fluorescence than the control animals. Fluorescence in the control animals is observed to spread throughout the peritoneal cavity, including the colon, cecum, small intestine, spleen, and peritoneal wall. GFP fluorescence is used to track tumor spread; numerous micrometastases are detected on the lungs of all control mice and multiple micrometastasis are also detected on the liver, kidney, contralateral ovary, adrenal gland, para-aortic lymph node, and pleural membrane.

1. A method to visualize a fluorescent protein through the skin of an intact subject, which method comprises applying excitation light to said subject using a portable light source with an attached first filter and observing emission from said protein through a second filter.

2. The method of claim 1, wherein the portable light source is an LED flashlight.

3. The method of claim 1, wherein the second filter is provided as a goggle.

4. The method of claim 1, wherein said fluorescent protein is expressed in tumor cells.

5. The method of claim 4, wherein said tumor cells are orthotopically implanted in an immunocompromised or syngeneic animal.

6. The method of claim 1, wherein the fluorescent protein is expressed by an infectious agent.

7. The method of claim 1, wherein the fluorescent protein is operatively linked to the control system for a gene whose expression is to be studied.

8. The method of claim 1, wherein observation is made of more than one fluorescent protein.

9. A method for monitoring tumor growth in an intact subject comprising:
applying excitation light using a portable excitation source with a first filter to a subject comprising tumor cells labeled with fluorescent protein; and
observing the location(s) of said tumor cells in the intact subject using a second filter.

10. The method of claim 9, wherein said applying of excitation light and observing are conducted as a function of time.

11. The method of claim 9, which further comprises treating said subject with a candidate protocol and comparing the location(s) of tumor cells in said subject treated with the protocol with the location(s) of tumor cells in a subject not treated with the protocol.

12. A method for monitoring gene expression in an intact subject which method comprises
applying an excitation light from a portable excitation source with a first filter to a subject comprising a nucleotide sequence encoding a fluorescent protein operably linked to control sequences associated with a gene whose expression is to be monitored and
observing the presence or amount of fluorescent protein in the intact subject using a second filter.

13. The method of claim 12, wherein said applying of excitation light and observing are conducted as a function of time.

14. The method of claim 12, which further comprises providing a stimulus to said subject and comparing the level of fluorescence emitted by the subject provided the stimulus to that emitted by a subject not provided with said stimulus.

15. A method to monitor the progress of infection in a subject which method comprises
applying excitation light using a portable excitation source with a first filter to a subject comprising an infectious agent labeled with fluorescent protein, and
observing the location(s) of said infectious agent in the intact subject using a second filter.

16. The method of claim 15, wherein said applying of excitation light and observing are conducted as a function of time.

17. The method of claim 15, which further comprises treating said subject with a candidate protocol and comparing the location(s) of infectious agent in said subject treated with said protocol with the location(s) of infectious agent in a subject not treated with the protocol.
